(19) United States
(12) Patent Application Publication
(10) Pub. No.: US 2022/0160966 A1
(43) Pub. Date: May 26, 2022

(54) SYRINGE BARREL WITH IMPROVED SCALE PRESENTATION

(71) Applicant: Nicholas William Halbach, Hayward, CA (US)

(72) Inventor: Nicholas William Halbach, Hayward, CA (US)

(21) Appl. No.: 17/670,494

(22) Filed: Feb. 13, 2022

Related U.S. Application Data
(63) Continuation-in-part of application No. 15/865,283, filed on Jan. 9, 2018, now Pat. No. 11,246,989.

(51) Int. Cl. A61M 5/31

(52) U.S. Cl. CPC A61M 5/3129; A61M 2202/0007; A61M 2005/3126

(57) ABSTRACT

A medical syringe with a modified barrel employs a graduated scale presentation for improved readability. The syringe's barrel has an outward and longitudinally aligned projection with a surface contiguous to the barrel's surface. The graduated scale is presented in whole or in part on the projection's surface, and is parallel to and immediately adjacent to the barrel's surface for edge-to-edge alignments with the fiducial line of the barrel's plunger. The graduated scale and its elements are compliant with ISO standards. Alternative scale presentations are readily adaptable for ISO consideration and expanded standards.

### CROSS-REFERENCE TO RELATED APPLICATION

This application is a continuation-in-part of application Ser. Nr. 15/865,283, Filed Jan. 9, 2018, now U.S. Pat. No. 11,246,989, Granted Feb 15, 2022.

### BACKGROUND

### Prior Art

FIGS. 1A to 1C (PRIOR ART) show three elevational views of a conventional plunger injection syringe assembly 100, with each view taken at a different angle of rotation. FIG. 1A: syringe assembly 100's main body aspect comprises a transparent barrel 101 with a side wall 101a defining a cylindrical lumen which is open at its proximal end and terminates as in communication with a lumen of a nozzle 102 at the barrel's distal end. A needle hub assembly 103 comprising a hollow injection needle 103a mounted to a hub 103b attaches onto nozzle 102, thereby connecting the lumens of needle 103a and barrel 101. A needle cap (not shown) covers hub assembly 103. A finger flange 104 is positioned at barrel 101's proximal end. The lumen of barrel 101 is occupied by an elongate plunger assembly comprising a slidable elastomeric plunger stopper 105 attached to a distal end of a plunger rod 106 which terminates proximally as a push button or bottom flange 106a. Proximal to flange 104, some syringes provide an end cap holder 107 as a mount for a plunger cap (not shown) which closes the lumen of barrel 101 with a plunger assembly fully retracted within, pre-use.

A surface of barrel 101 typically bears a vertical column of graduation lines 108, 109, 110 (FIG. 1C), and correlated numerical markings 111 (FIG. 1B)—collectively comprising a graduated scale 300 which guides the user in drawing a measured dose of the medicament fluid into the lumen of the barrel. An upper edge or leading flange seal 105a of plunger stopper 105 serves as a fiducial line that, in cooperation with overlying scale 300 on barrel 101, defines a dose selection.

Syringe 100 is an exemplary 30-unit, single-use insulin syringe, calibrated for use with U-100 (100 units/ml) insulin, and is marked in ½ unit increments. I.e., scale 300 can measure up to 30 units or 0.3 ml of insulin, with each whole unit on the scale representing 0.01 ml. As a working example, in FIGS. 1A to 1C leading flange seal/fiducial line 105a of plunger stopper 105 is at unit 7 of scale 300, and the dose selected by that plunger position is seven units or 0.07 ml of insulin. With a nominal (i.e., the manufacturer's designated) capacity of 0.3 ml, syringe 100 is one of the smallest medical syringes in mass production and general use.

In field use the conventional presentation of graduated scale 300 on cylindrical barrel 101 poses notable disadvantages, particularly on miniaturized syringes with thin barrels, e.g., insulin syringes. As barrel 101 has a very small surface area, the font size of numerical markings 111 must be commensurately small. The limited surface of barrel 101, coupled with its narrow width and sharp radial curvature, renders merely partial and distorted/horizonal views of scale 300. Thus, as shown in series in FIGS. 1A to 1C, a user must repeatedly rotate the syringe side-to-side in an axial manner to fully visualize and assimilate the graduation lines and numbers on scale 300 as they orient the syringe to draw an indicated dose. The crowded and dense column of scale 300's markings impedes viewing of the thin barrel's fluid contents, including aberrant air gaps and bubbles therein. Another visual impediment is the manner in which plunger stopper 105 continually obscures superimposed scale 300 as the user adjusts the plunger for the final desired draw point. Diabetic patients commonly suffer from vision impairments, compounding the above issues.

Due to the intrinsic challenges of insulin syringes—and their global use at the home-patient level—the International Organization for Standardization (ISO), headquartered at Geneva Switzerland (website: www.iso.org), oversees, regularly reviews, and publishes detailed criteria and specifications specifically for insulin syringes, including presentation of the graduated scale. The resultant ISO-formulated standard, ISO 8537, is universally adopted by syringe manufacturers, independent testing laboratories, and regulatory domains (e.g., the FDA) in the development, review, approval, and ultimate registration of insulin syringes as medical devices.

FIG. 2 (PRIOR ART) diagrams the following key parameters for a graduated scale 300 of an insulin syringe under the current ISO standard:
A—longitudinal length of scale 300;
B—scale intervals in whole units (may be further refined to 0.5-unit intervals);
C—major unit interval;
D—vertical thickness of unit graduation lines 109, 110;
E—transverse width/span of a minor unit graduation line 109 (relative to a major line 110);
F—transverse width/span of a major unit graduation line 110 (relative to a minor line 109);
G—spacing between a major unit graduation line 110 and its numerical marking 111;
H—orientation of graduation lines relative to the syringe barrel axis;
I—height of a numerical marking 111; and,
J—orientation and positioning of a numerical marking 111 relative to other elements.

Additional elements, parameters, criteria, and specifications thereof for scale and other syringe indicia presentation may apply, including specified insulin concentrations of use (in print and tactile form), other text, numbers, volumetric and unit denotations, and symbols.

Within that scope, ISO's objectives are to ensure optimal visibility, readability, and useability of the graduated scale—within the inherent limitations of conventional insulin syringe size and arrangement. Beyond that scope ISO also encourages innovation and improvements evolution for their consideration.

Thus, it would be desirable to render graduated scale presentation for insulin syringes that: mitigates the deficiencies of conventional presentation methods; is in accordance and compliance with existing ISO standards; possesses latitude for advancing ISO standards—within the envelope of ISO review, development, and approval; and possesses latitude for compliance with any additional mandates by a regulatory body in registration as a medical device.

### SUMMARY

Several embodiments of syringes employ a modified syringe barrel comprising an elongate main body portion with a side wall defining cylindrical lumen, and an outward projection longitudinally aligned with and laterally adjacent to the side wall. Part or all of a columnar graduated scale is presented on the projection, parallel with and immediately adjacent to the side wall. This arrangement renders a substantially flat scale presentation which is more readable and has more incisive alignments with the plunger's fiducial line, and greater visibility of the barrel's contents. The collective scale and its individual elements embody, and are in accordance and compliance with, current ISO specifications and standards for a conventional insulin syringe. Other alternative scale presentations shown have latitude for ISO consideration and expanded standards within the envelope of the ISO review process. Furthermore, the novel arrangements are adaptable for rendering scale presentations for other non-insulin syringes which are registrable as medical devices. Further advantages of one or more aspects may become apparent from a consideration of the accompanying drawings and ensuing description.

### DRAWING FIGURES

FIG. 1A (PRIOR ART) is a photo of a conventional 30-unit insulin syringe and its graduated scale.

FIG. 1B (PRIOR ART) is a photo of a rotated view of the syringe in FIG. 1A.

FIG. 1C (PRIOR ART) is a photo of a further rotated view of the syringe in FIG. 1A.

FIG. 2 (PRIOR ART) is a general schematic of parameters for the ISO standard for a graduated scale of an insulin syringe.

FIG. 3A shows an elevational view from slightly above of a syringe barrel with a projection on the barrel.

FIG. 3B shows a sectional view of the syringe of FIG. 3A taken along the line 3B-3B.

FIG. 4 shows a similar elevational view from slightly above of a syringe with a first version of an alternative graduated scale presentation, using the barrel of FIG. 3A as a platform.

FIG. 5 shows an elevational view from slightly above of a syringe with a second version of an alternative graduated scale presentation, using the barrel of FIG. 3A as a platform, similar to that of FIG. 4.

FIG. 6A shows an elevational view from slightly above of a third version of an alternative graduated scale presentation, using another alternative insulin syringe as a platform, also similar to that of FIG. 4.

FIG. 6B shows a sectional view of the syringe of FIG. 6A taken as indicated by the section lines 6B-6B.

FIG. 7 shows an elevational view from slightly above of a syringe barrel with a fourth version of an alternative graduated scale presentation, using the barrel of FIG. 3A as a platform.

FIG. 8 shows an elevational view from slightly above of a syringe barrel with a fifth version of an alternative graduated scale presentation, using the barrel of FIG. 3A as a platform.

FIG. 9A shows an elevational view of another alternative syringe with a sixth version of an alternative graduated scale presentation, using the barrel of FIG. 3A as a platform.

FIG. 9B is an enlarged view of an aspect of the syringe in FIG. 9A, to illustrate a whole unit dose selection of insulin.

FIG. 9C is an enlarged view of an aspect of the syringe in FIG. 9A, to illustrate a half unit dose selection of insulin.

FIG. 10 shows an elevational view from slightly above of an alternative syringe with a 0.1 ml/10-unit insulin capacity, and a seventh version of an alternative graduated scale presentation.

FIG. 11 shows an elevational view from slightly above of an alternative syringe with a 0.1 ml/10-unit insulin capacity, and an eighth version of an alternative graduated scale presentation.

FIG. 12 shows an elevational view from slightly above of an alternative syringe with a 0.1 ml/10-unit insulin capacity, and a ninth version of an alternative graduated scale presentation.

FIG. 13 shows an elevational view from slightly above of PRIOR ART safety syringe, modified with a lateral projection and scale display thereon.

FIG. 14 shows an alternative syringe with a non-needled nozzle ejection end, and a tenth version of an alternative graduated scale presentation.

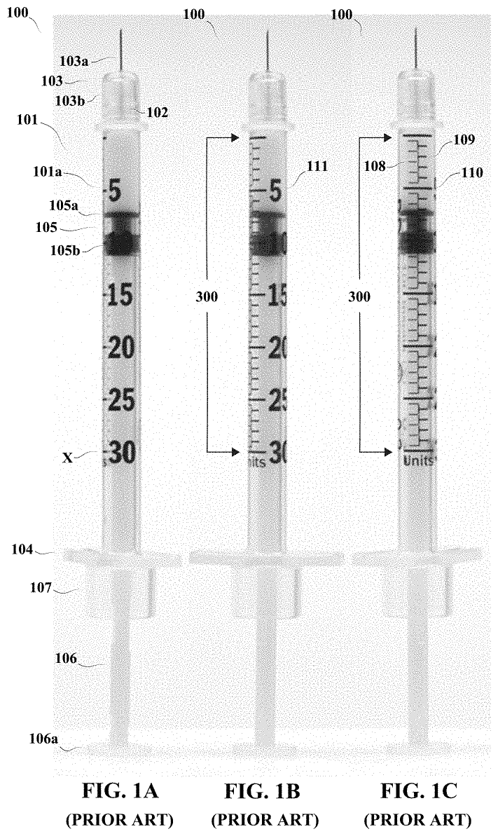

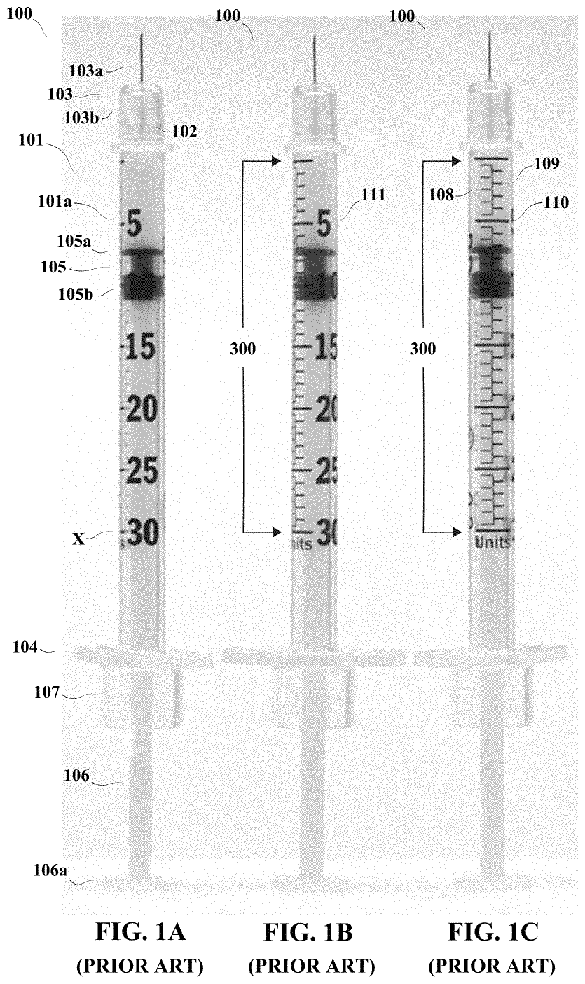

### REFERENCE CHARACTERS

A: longitudinal length of scale (also denotes nominal capacity)
B: space between unit graduation lines
C: major unit interval
D: thickness of unit graduation lines
E: transverse width/span of a minor unit graduation line relative to a major line
F: transverse width/span of a major unit graduation line relative to a minor line
G: space between a major unit graduation line and its numerical marking
H: orientation of graduation lines relative to the syringe barrel axis
I: height of a numerical marking
J: orientation of a numerical figure relative to other elements
X: syringe nominal capacity/nominal capacity line of graduated scale
Z: zero line of graduated scale
100—exemplary conventional 30 unit/0.3 ml insulin syringe
100a-100h first to eighth syringe embodiments
101—barrel of conventional syringe
101a—side wall of 101
102—nozzle
103—needle hub assembly
103a—hollow injection needle
103b—hub
104—finger flange
105—plunger stopper
105a—leading flange seal/fiducial line of plunger stopper
105b—backup flange seal of plunger stopper
106—plunger rod
106a—bottom flange of plunger rod
107—end cap holder
108—spacing for indicating ½ unit of insulin
1096—minor graduation line for one unit of insulin
110—major graduation line for units of insulin
111—numerical marking or number for units of insulin
201—modified syringe barrel with lateral projection
201a—side wall of barrel 201
201b—lumen of barrel 201
300—conventional graduated scale presentation
301-310 first to tenth graduated scale presentations
400—lateral projection
402—adhesive label
404—lateral projection (paper, coated paper, or thin polymer)
404a—adhesive aspect of lateral projection 404
500—non-needled dispensing nozzle

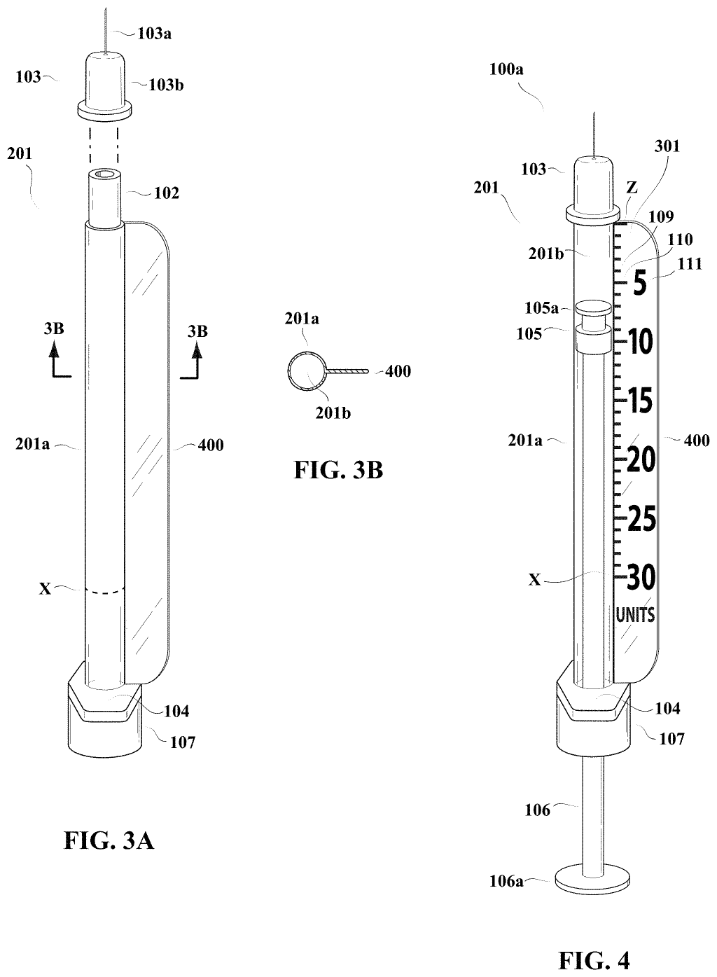

### DETAILED DESCRIPTION—BASIC PLATFORM—FIGS 3A and 3B

FIGS. 3A and 3B show a modified syringe barrel 201 with a projection 400. Barrel 201 provides a basic and constant platform of the syringes of FIGS. 4 et seq., and is shown in an exemplary nominal capacity of 0.3 ml or 30 units of U-100 insulin. A main body portion of barrel 201 comprises an elongate, transparent side wall 201a surrounding and defining a cylindrical lumen 201b, with an open proximal end and a distal end terminating with a passageway to a lumen of a fore-extension, nozzle 102. At assembly, a needle hub assembly 103 comprising a hollow injection needle 103a mounted to a hub 103b is attached onto nozzle 102, thereby connecting the lumens of needle 103a and barrel 201 (some conventional insulin syringe constructs connect needle 103a directly to nozzle 102, sans hub 103b). Other extensions to barrel 201 include a finger flange 104 proximally, and an optional end cap holder 107.

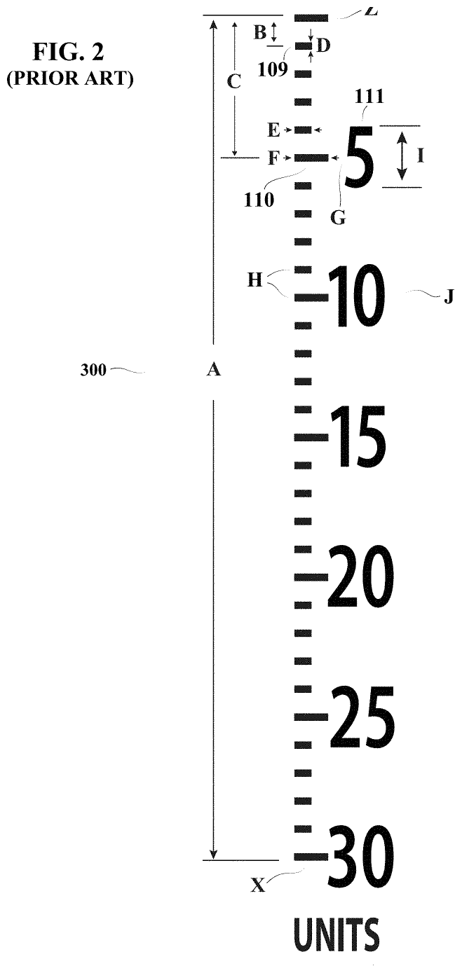

Barrel 201's side wall aspect 201a is presented herein as being in accordance and compliance with the current ISO Standard 8537 for an insulin syringe with an exemplary nominal capacity X of 0.3 ml or 30 units of U-100 insulin. More specifically, wall 201a's length is such that the syringe has a usable capacity of either 10% more than its nominal capacity X or 3 mm of plunger travel beyond the nominal capacity graduation line X, whichever is less (“usable capacity” is defined as the capacity of the syringe when the plunger stopper is drawn proximally to its furthest functional position beyond the nominal capacity graduation line X). The length of wall 201a's nominal capacity aspect is dictated by the ISO standard for the graduated scale length A (FIG. 2) of a 0.3 ml/30-unit syringe-41 mm.

A projection 400 (or alternatively, a flexible projection 404) is provided on side wall 201a's surface. Projection 400 is an oblong and tab-like projection longitudinally aligned with wall 201a, and it extends laterally outwardly and away from wall 201a in a single general direction. Projection 400 extends longitudinally to or beyond a distal limit of barrel 201's lumen 201b. Projection 400 provides an off-barrel major front face with a surface that is contiguous along a continuous inner margin or line with the outer surface of wall 201a, thereby providing an immediately adjacent auxiliary surface area for bearing or displaying part or all of the elements of a graduated scale and other indicia. Projection 400 also has a rear face with a surface for indicia. Projection 400 is rendered with longitudinal and transverse dimensions for full display, including outer margins, of any graduated scale elements to be presented thereupon. In FIG. 3A projection 400 is shown extending the entire longitudinal length of wall 201a to finger flange 104 for added surface proximally and structural reinforcement of wall 201a; shorter length projections are possible. Dimensions for that new element, projection 400/404, may be further defined under expanded ISO parameters, specifications, and standards.

For clarity, in all descriptions herein the term “graduated scale” and its collective elements refers specifically to a columnar array of graduation lines and their associated numerical figures, with or without any additional elements or indicia—e.g., text, numbers, metric denotations, symbols, or other markings that may be indicated for a registrable insulin syringe. The term “scale presentation” refers to the physical arrangement or presentation of the graduated scale.

### First Embodiment

### FIG. 4—Rigid Projection with Printed or Molded Indicia

FIG. 4 shows a first syringe assembly 100a, incorporating modified syringe barrel 201 above (FIGS. 3A, 3B) with its lateral projection 400 on side wall 201a. The nominal capacity X of syringe 100a is shown as 30 units or 0.3 ml, and is for use with U-100 insulin.

A first graduated scale presentation 301 is shown on projection 400's major front face, spanning from a zero-line X at barrel 201's distal luminal limit to the nominal capacity line X at 30 units (plus an added margin of length to include the scale's last numerical marking, “30”). Scale presentation 301 embodies minor and major unit incremental graduation lines 109 and 110, and ascending numerical markings 111 adjacent every fifth (major) graduation line, and it displays those elements as a columnar array on a surface of projection 400—parallel with, directly adjacent to, and entirely off of the surface of barrel 201's side wall 201a. An inner margin of the column of lines 109 and 110 abuts the surface of wall 201a for visual edge-to-edge alignment with a fiducial line or leading flange seal 105a of a plunger stopper 105. Thus, in operation a desired dose is measured by alignment of seal 105a with a corresponding graduation line on projection 400; FIG. 4 shows a measurement of seven units of insulin.

Moreover, in FIG. 4 scale presentation 301 sizes and arranges scale elements 109, 110, and 111 in accordance with specifications of the ISO 8537 standard for a conventional 30-unit insulin syringe marked in one-unit intervals. Those specification parameters are shown in FIG. 2. Accordingly, projection 400 is rendered in transverse and longitudinal dimensions for accommodating display of scale presentation 301, again, subject to any expanded ISO parameters and specifications for that new extension.

Fabrication of syringe 100a and its parts is achieved by suitable technologies for a polymer syringe. Projection 400 in this embodiment is of a polymer having a sufficient thickness to be rigid and robust, and is preferably integrally molded with side wall 201a of barrel 201. By its thickness projection 400 may also add collateral support to side wall 201a, and thus possibly enable a thinner and more transparent wall 201a, for higher visibility of lumen 201b. The elements of scale 301 can be applied to projection 400 by any of a number of suitable technologies, including printing, embossing, and integral molding—and/or including tactile or raised presentations to assist the visually impaired. Any additional indicia, including text, numbers, volume and unit denotations, symbols, or other markings, can be sized and positioned on projection 400 (front or rear surfaces) and/or barrel side wall 201a for optimal viewability. ISO directives for manufacturer risk analysis, usability testing, and validation testing should be followed in fulfilling these developments and refinements.

Accordingly, the reader will see that the above exemplary embodiment demonstrates significant advantages over conventional insulin syringe graduated scale presentations. A scale can be arranged and positioned with some of the scale's elements on the barrel side wall and some elements on the projection. Or, as shown in FIG. 4, all of the scale's elements can be arranged and positioned on the projection and entirely off of the barrel wall, thereby flattening, simplifying, and clarifying the scale's viewability, while enabling unobstructed viewability of the contents of the barrel. The direct side-by-side, edge-to-edge orientation and alignment of the plunger stopper's fiducial line with a scale graduation line offers a much sharper and more incisive dose selection view to the user. Clearing the syringe barrel of graduated scale markings gives the user an unimpeded view of the barrel's fluid/insulin contents, aiding in detection and resolution of aberrant air gaps and bubbles. The collective effect is an easier patient experience, with reduced insulin dosing error.

### Second Embodiment

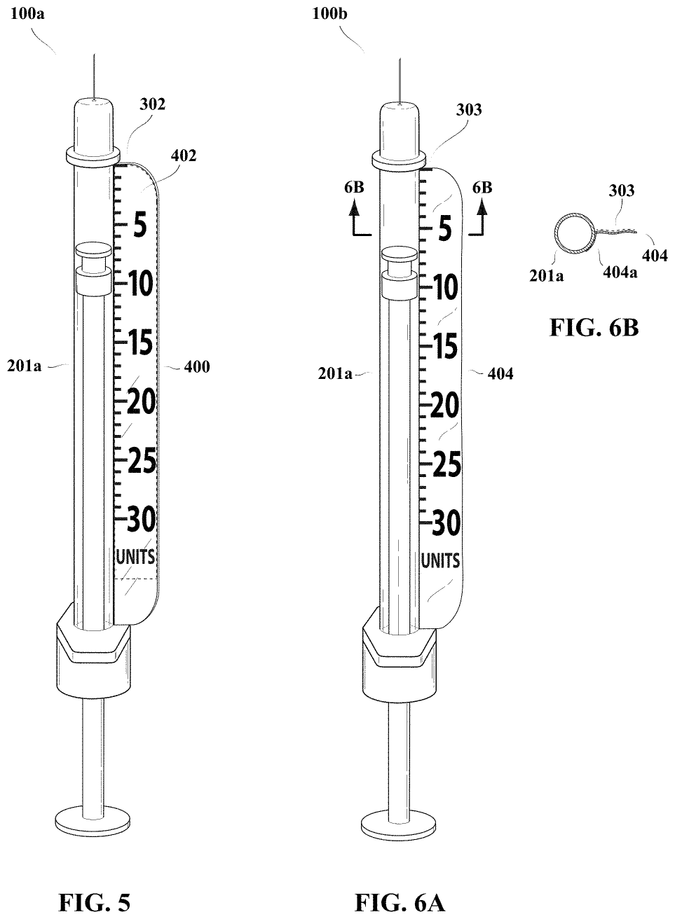

### FIG. 5—Indicia Provided on Label

FIG. 5 shows a second graduated scale presentation 302, again using as a platform the modified insulin syringe 100a above (FIG. 4) with its lateral projection 400 on barrel side wall 201a. Here scale presentation 302 utilizes the same ISO-compliant configuration of graduation lines and numerical figures as presentation 301 of FIG. 4, with the exception that these elements are printed in high resolution onto a label 402 (edge shown by a dashed line) which is then affixed to the surface of projection 400 by a suitable adhesive using well-known techniques. E.g., the adhesive can be applied to the back of the label and covered by a release sheet which can be peeled off to expose the adhesive for application onto the projection. Alternatively, the adhesive can be applied to the projection and covered by a release sheet, or the projection and the label, prior to application, using conventional techniques.

### Third Embodiment

### FIGS. 6A, 6B—Flexible Projection

FIGS. 6A and 6B show isometric and cross-sectional views of a third graduated scale presentation 303, using as a platform a second modified 30-unit insulin syringe 100b. Here a longitudinal aligned and laterally outward tab-like projection 404 on a polymer barrel side wall 201a is provided as an elongate strip comprised of paper, coated paper, foil, polymer sheet, or of other thin materials—alone or in composite. This strip is flexible and not rigid or as robust as the projections of the previous embodiments, but is adequate for its purpose of providing a highly-visible quantity-indicating scale for a single-use or disposable syringe. Scale presentation 303 is displayed on a surface of projection 404, using the same ISO-compliant configuration as presentations 301 and 302 above. Projection 404 is attached to wall 201a via an adhesive-backed area 404a extending from an inner longitudinal aspect of projection 404, with projection 404 being folded accordingly at its junction with wall 201a for direct side-by-side viewing with scale presentation 303. Scale presentation 303 utilizes the same ISO-compliant configuration of graduation lines and numerical figures as first and second scale presentations 301 and 302 above.

Shown above in 30 unit/0.3 ml nominal capacities with one-unit scale intervals, and for use with U-100 insulin, exemplary syringes 200a, 200b, and graduated scale presentations 301, 302, and 303 are adaptable for other nominal capacities (e.g., 0.5 ml, 1.0 ml, 2.0 ml), other scale unit intervals (e.g., 0.25, 0.5 units, or other), and other standard insulin concentrations (e.g., U-40, U-200, U-300, or U-500). Projections 400 and 404 and scale display methods shown can also be integrated with other insulin syringe constructs, including those with specialized safety features. Any such variants would also be rendered in compliance with the applicable ISO Standard 8537.

Exemplary graduated scale presentations 301, 302, and 303 above are described and illustrated as being technically in full compliance with the current ISO specification 8537 thereof for a conventional insulin syringe. However, the complete off-barrel positioning of those scales, and the novel syringe structures 100a and 100b supporting those presentations are not fully addressed by the present ISO standard. Thus, collectively for those innovations—i.e., syringes modified with barrel projections 400 or 404 and the methods for alternative scale presentation thereon—validation testing and reviews for risk, safety, and usability would apply, e.g., under ISO 14971, ISO 13485, and IEC 62366. If ultimately certified by ISO, by extension those innovations would have effectively generated their own unique/novel set of parameters and specifications within an (expanded) ISO 8537 standard.

### Description—Alternative Embodiments

The following alternative graduated scale presentations for insulin syringes range beyond the scope of current ISO specifications, but are readily adaptable for consideration within the envelope of ISO review. As per directly above, for such innovations supporting studies for risk, safety, and usability may also be requisite to ISO certification

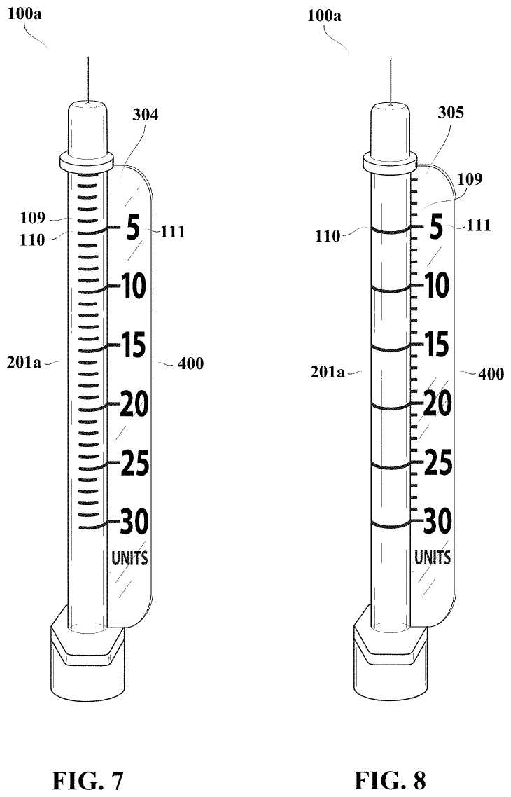

Fourth Embodiment: FIG. 7

### Graduated Scale Presented on Barrel and Projection

FIG. 7 shows a fourth graduated scale presentation 304, again, using as a platform modified insulin syringe 100a (FIG. 4) with its lateral projection 400 on barrel side wall 201a. Configured with one-unit intervals, presentation 304 places the column of minor 109 and major 110 graduation lines on the surface of wall 201a and it extends the major graduation lines 110 laterally onto projection 400 to lead to correlating numerical FIGS. 111.

### Fifth Embodiment: FIG. 8

### Graduated Scale Presented on Barrel and Projection

FIG. 8 shows a fifth graduated scale presentation 305, again using as a platform syringe 100a with its lateral projection 400 on barrel side wall 201a. Configured with one-unit intervals, presentation 305 places the column of minor 109 and major 110 graduation lines and numbers 111 on the surface of projection 400, and it extends the major graduation lines 110 onto wall 201a.

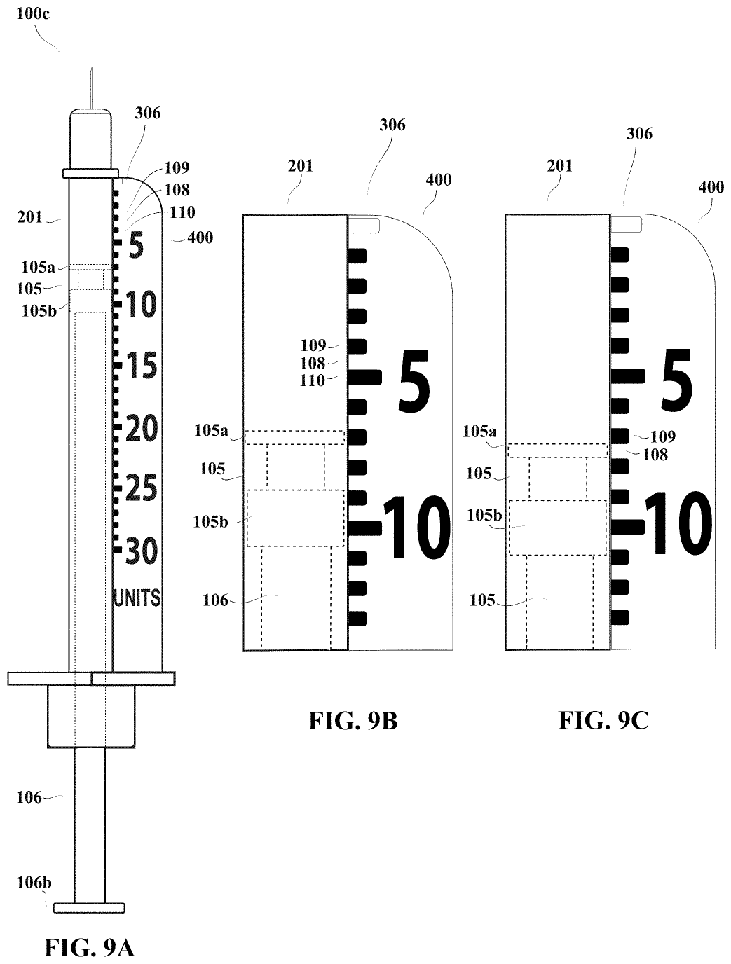

### Sixth Embodiment: FIGS. 9A-9C

### Plunger Flange Thickness Corresponds to Graduation Line

FIG. 9A shows another insulin syringe assembly 100c which is identical to syringe 100a above, with the exception that plunger stopper 105 has a leading flange seal 105a with a vertical thickness that assumes a specific vertical dimension when compressed within barrel 201. Syringe assembly 100c provides the platform for a sixth graduated scale presentation 306, as follows. As shown in FIG. 9A and in more detail in FIGS. 9B and 9C, plunger stopper 105 has a compressible leading flange seal 105a and a backup seal 105b. Note (FIG. 9B) that leading seal 105a, when compressed within barrel 201, has the same thickness or vertical dimension as graduation lines 109 and 110 and spacings 108 between the lines. In operation, a whole-unit dose of insulin is selected by full alignment of leading annular seal 105a with a graduation line 109, 110; seven units are shown selected in FIG. 9B. Half-unit measurements are selected by full alignment of seal 105a with a spacing 108 between graduation lines; 7.5 units are shown selected in FIG. 9C. This method of scale presentation 306 simplifies nearest half-unit insulin dosing over conventional scales, and is adaptable to other capacity insulin syringes and other types of syringes with different dosing metrics.

Whole the above examples show modified 0.3 ml/30-unit capacity syringes for use with U-100 insulin. Those syringes and methods of graduated scale presentation are adaptable for other insulin syringe nominal capacities, for other scale unit intervals, and for other insulin concentrations. Any combination of modified syringe-100a, 100b, 100c, or graduated scale presentation 301-306 is possible in rendering an improved scale presentation. Moreover, those principles are adaptable for other types of medical syringes and other capacities—with or without attached needles—for delivering other medicaments, and with calibration for dose measurement via graduated scales with other metrics. Furthermore, those principles can be used to render syringes for non-medical applications.

The lowest nominal capacity of a conventional insulin syringe is currently 0.3 ml/30-units when using U-100 insulin. The alternative syringes as modeled above, with their off-barrel scale display capabilities and alternative display methods, enable even lower nominal capacities not previously attainable for insulin syringes, as shown in the following three examples:

### Seventh Embodiment

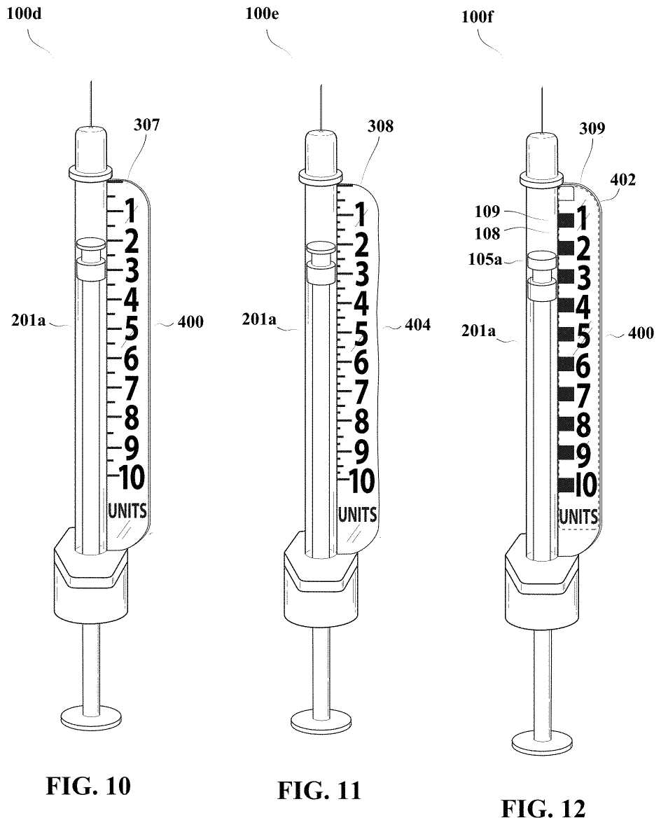

### FIG 10: 10 Unit Syringe and Scale

FIG. 10 shows a fourth insulin syringe 100d with a nominal capacity of 0.1 ml/10 units, with a projection 400 on barrel side wall 201a. A seventh graduated scale or presentation 307 configured with full and half-unit graduations is displayed on projection 400.

### Eighth Embodiment

### fIG 10: 10 Unit Syringe and Scale with Quarter Unit Graduations

FIG. 11 shows a fifth insulin syringe 100e with a nominal capacity of 0.1 ml/10 units, with an eighth graduated scale or presentation 308 bearing nearest quarter-unit graduations on a thinner, less rigid projection 404 adhered to barrel side wall 201a, as described above for larger syringe 100b in FIGS. 6A and 6B.

### Ninth Embodiment

### FIG 12: 10 Unit Syringe with Plunger Flange Thickness Corresponding to Graduation Line

FIG. 12 shows a sixth insulin syringe 100f with a nominal capacity of 0.1 ml/10 units, a projection 400 on barrel side wall 201a, and a ninth graduated scale presentation 309 on projection 400. Syringe 100f and scale presentation 309 employ the same leading flange seal 105a which has the same thickness as unit graduation lines 109 and spacing 108 between lines as described for syringe 100c and scale 306 in FIGS. 9A-9C. This arrangement facilitates measuring dose draws down to a half-unit accurately; 2½ units are shown measured. In FIG. 12 graduated scale presentation 309 is also shown on an adhesive label 402 (edge shown by dashed line), which is then applied to projection 400, as described above for second scale 302 in FIG. 5.

The 0.1 ml/10-unit insulin syringes above in FIGS. 10-12, with their relatively elongated and refined graduated scales, will be particularly advantageous for critical precision dosing (e.g., ½ or ¼-unit increments) with small patients, e.g., in pediatric and veterinary applications. As with larger embodiments above, various permutations of projection construction and scale presentation are possible from among the examples shown. Again, neither these 10 unit capacity syringes or their graduated scale presentations/methods are currently described or specified under the ISO 8537 standard. Thus, as with other embodiments above, validation testing, followed by expanded ISO standards would be indicated. A range of non-insulin adaptations with other metrics are also possible for these ultra-low-capacity syringes—e.g., human and veterinary medicine, dentistry, dermatology, and research/lab animal medicine.

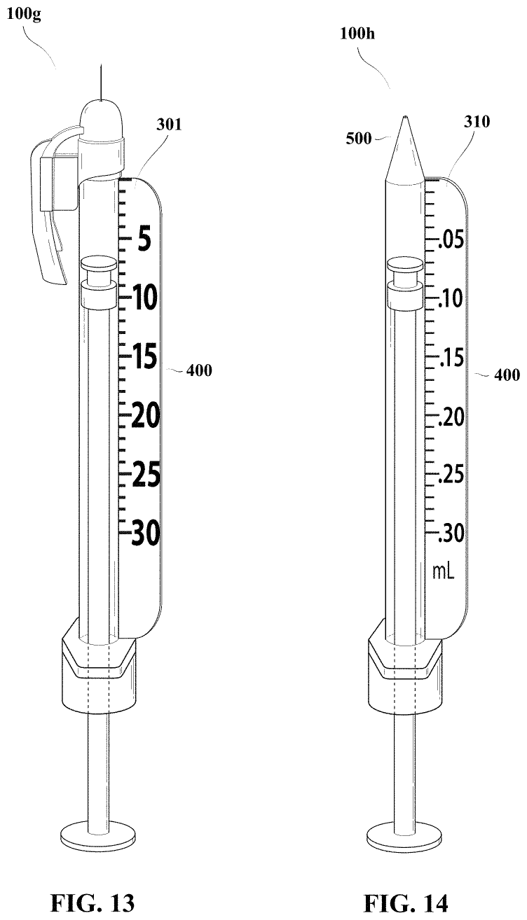

Projection 400 and alternative scale presentation methods shown herein can be integrated with other syringe constructs. For example, FIG. 13 shows an existing (PRIOR ART) safety insulin syringe which has been modified as a seventh insulin syringe 100g to include a projection 400 bearing first graduated scale presentation 301. FIG. 14 shows an eighth syringe 100h with a non-needled dispensing nozzle 500, and a tenth graduated scale presentation 310 which has calibration in nearest 0.01 ml increments.

### CONCLUSIONS, RAMIFICATIONS, AND SCOPE

Accordingly, the reader will see that above syringe barrels and graduated scale presentations demonstrate significant advantages over conventional insulin syringes.

A scale can be configured and positioned partially or completely off-barrel onto an adjacent projection, thereby flattening, simplifying, and clarifying the scale's viewability. Direct side-by-side, edge-to-edge alignment of the plunger's fiducial line with a scale line yields more incisive dose selection. Clearing the syringe barrel of scale markings gives the user an unimpeded view of the barrel's fluid/insulin contents, aiding in detection and resolution of aberrant air gaps and bubbles. Diabetic patients commonly suffer from vision loss. Modified syringes and scale presentations herein address their challenges with optimized visibility, precision, and, ultimately, reduced dosing error for higher safety in insulin use.

FIGS. 4 to 6B show syringe barrels with alternative scale presentations that render the above in full compliance with current ISO specifications for an insulin syringe. Within that scope those presentation methods are adaptable to the full range of insulin syringe nominal capacities, insulin unit/ml concentrations, scale unit intervals, and specialized syringes—including those with safety needles.

ISO encourages innovation, and the devices and methods of this application propose as much for consideration and inclusion within the domain of ISO 8537. FIGS. 7 to 13 show other alternative syringe barrels/insulin syringes and scale presentations that are readily adaptable for utility under expanded ISO standards. Validation testing for risk, safety, and usability would be requisite to an ISO review. As new features, that might include projections 440 and 404—more specifically, their transverse and longitudinal dimensions, plus any outer margins, as stages for various scale presentations by methods shown herein. In turn, collectively those modifications would add their own set of unique parameters and specifications to ISO 8537. Ample surface area—front and rear—of projections 400/404 offers generous latitude for presentation of all syringe indicia in bolder, clearer manners, including pre-printed, high resolution label formats, or raised, tactile elements. Thus, it becomes possible to advance and exceed present ISO specifications, including more prominent graduation lines and numerals, and to introduce ultralow nominal capacities not possible with conventional syringes and scale presentation methods (FIGS. 10-12). From among the above embodiments, various combinations and permutations of projection structure and scale presentation are possible for rendering an improved insulin syringe.

Similarly, the embodiments' various features—alone or in combination—are adaptable to a wide range of other (non-insulin) medical syringes for other uses and medicaments, with other nominal capacities, and with graduated scales configured for other metrics. That may include syringes with attached needles, safety needle extensions and apparatuses, open hub ends for needle attachment, or other non-needled exit ports. Potential utilities include therapeutics and diagnostics for human and non-human animals, dermatology and dentistry applications, medicament compounding, and laboratory/research settings. Separate ISO standards may apply for such syringes, along with appropriate pre-regulatory testing and validations.

Furthermore, the barrel and scale presentation modifications herein are implementable within syringes for a broad range of non-medical utilities, including laboratory, manufacturing, and consumer areas.

Thus, the scope of the embodiments should be determined by the ensuing claims and their legal equivalents, rather than by the examples given.

1. A modified barrel for a syringe comprising:
an elongate body portion having a side wall defining a cylindrical lumen, said lumen having a longitudinal axis, said body portion having an open proximal end and a distal end having a passageway for conveying contents in said lumen to a hollow nozzle or needle,
said side wall having an outer surface and a generally oblong projection in longitudinal alignment with said side wall and said body portion, said projection extending laterally outwardly and away from said outer surface of said side wall in a single general direction, said projection extending longitudinally to or beyond a distal limit of said lumen;
said projection having a front major face and a rear face, said front face being generally planar and being contiguous along a continuous inner longitudinal edge with said outer surface of said side wall; and,
at least one of said faces bearing at least part of a graduated scale;
said scale being in compliance with a predetermined standard for an indicating scale for indicating the quantity of medicament in said syringe barrel;
whereby said syringe is in compliance with said predetermined standard and provides a more readable display.

2. The barrel of claim 1 wherein said graduated scale comprises a plurality of incremental line markings and numerical markings.

3. The barrel of claim 1 wherein said predetermined standard is by the International Organization for Standardization.

4. The barrel of claim 3 wherein said at least one of said faces has sufficient dimensions for displaying a graduated scale in accordance with said standard by the International Organization for Standardization.

5. The barrel of claim 1 wherein a part of said graduated scale appears on said outer surface of said side wall.

6. The barrel of claim 1 wherein said barrel is for an insulin syringe.

7. A barrel for an insulin syringe comprising:
an elongate body portion having a side wall defining a cylindrical lumen with a longitudinal axis, said body portion having an open proximal end and a distal end having a passageway therethrough in communication with said lumen;
said side wall having an outer surface and a generally oblong projection in longitudinal alignment with said side wall and said body portion, said projection extending laterally outwardly and away from said outer surface of said side wall in a single general direction, said projection extending longitudinally to or beyond a distal limit of said lumen;
said projection having a front major face and a rear face, said front face being generally planar and being contiguous along a continuous inner longitudinal edge with an outer surface of said side wall; and,
at least one of said faces bearing at least part of a graduated scale,
said barrel being in compliance with a predetermined legal standard for an insulin syringe, and
said scale being in compliance with a predetermined legal standard for an indicating scale for indicating the quantity of medicament in said syringe barrel,
whereby said syringe is in legal compliance with said predetermined standard and provides a more readable display.

8. The barrel of claim 7 wherein a part of said graduated scale appears on said outer surface of said side wall.

9. The barrel of claim 7 wherein said graduated scale comprises a plurality of incremental line markings and numerical markings.

10. The barrel of claim 7 wherein said predetermined standard is by the International Organization for Standardization.

11. The barrel of claim 9 wherein said at least one of said faces has sufficient dimensions for displaying a graduated scale in accordance with said predetermined standard by the International Organization for Standardization.

12. A method of providing a syringe with a more readable display, comprising:
providing an elongate body portion having a side wall having an outer surface and defining a cylindrical lumen, said lumen having a longitudinal axis, said body portion having an open proximal end and a distal end having a passageway for conveying contents in said lumen to a hollow nozzle or needle,
providing a projection extending laterally outwardly and away from said outer surface of said side wall in a single general direction, said projection extending longitudinally to or beyond a distal limit of said lumen, said projection being generally oblong and longitudinally aligned with said side wall and said body portion,
said projection having a front major face and a rear face, said front face being generally planar and being contiguous along a continuous inner longitudinal edge with said outer surface of said side wall; and,
at least one of said faces bearing at least part of a graduated scale;
said scale being in compliance with a predetermined standard for an indicating scale for indicating the quantity of medicament in said syringe barrel;
whereby said syringe is in compliance with said predetermined standard and provides a more readable display.

13. The method of claim 12 wherein said graduated scale is provided with a plurality of incremental line markings and numerical markings.

14. The barrel of claim 12 wherein said predetermined standard is by the International Organization for Standardization.

15. The barrel of claim 14 wherein said at least one of said faces has sufficient dimensions for displaying a graduated scale in accordance with said standard by said International Organization for Standardization

16. The barrel of claim 12 wherein a part of said graduated scale is provided on said outer surface of said side wall.

17. The barrel of claim 12 wherein said barrel is provided for holding and dispensing insulin.
